(19) United States
(12) Patent Application Publication
(10) Pub. No.: US 2016/0114182 A1
(43) Pub. Date: Apr. 28, 2016

(54) Electron Flow Detoxification System

(71) Applicant: Kenneth Evan Nicholson, Raeford, NC (US)

(72) Inventor: Kenneth Evan Nicholson, Raeford, NC (US)

(21) Appl. No.: 14/878,109

(22) Filed: Oct. 8, 2015

Related U.S. Application Data
(60) Provisional application No. 62/067,038, filed on Oct. 22, 2014.

(51) Int. Cl. A61N 1/44; A61N 1/20

(52) U.S. Cl. CPC A61N 1/445; A61N 1/20

(57) ABSTRACT

An electron flow detoxification system is a system for eliminating toxins and other harmful substances in the user's body. A quantity of saltwater is placed into a fluid reservoir. An electrode assembly is connected to a switching power supply and is submerged in the quantity of saltwater. The switching power supply produces an electrical current that enters the quantity of saltwater and into the user's bloodstream. The electrical current circulates through the user's bloodstream and upon exiting the user's body, draws toxins and other harmful substances from the user's body. The switching power supply is protected within an enclosure along with a control unit, an electrical relay set, and additional electronic components.

The current application claims a priority to the U.S. Provisional Patent application Ser. No. 62/067,038 filed on Oct. 22, 2014.

### FIELD OF THE INVENTION

The present invention relates generally to a detoxification system. More specifically, the present invention is an electron flow detoxification system for removing toxins and other harmful substances through the feet or hands.

### BACKGROUND OF THE INVENTION

Toxins and other harmful substances are able to build up over time in the body. Commonly consumed foods often contain a large amounts of unnecessary and possibly harmful ingredients such as flavor enhancers, food colorings, pesticides, and preservatives. Additionally, water, drugs, and ambient pollutants can all contribute to the accumulation of toxins that linger in the body, often for years. Various detoxification methods are available in order to remove impurities from the blood. The kidneys, intestines, lungs, and skin are also capable of removing and eliminating toxins. However, when this system becomes compromised or is ineffective, impurities in the blood cannot be properly filtered and the body is adversely affected. The present invention seeks to address the aforementioned issues as well as provide an alternative means removing toxins and other harmful substances from the body.

The present invention is an electron flow detoxification system that is able to draw toxins and other harmful substances from the user's body. The user is able to submerge his or her hands or feet into a fluid reservoir holding a quantity of saltwater. The quantity of saltwater serves as a conductor for an electrical current produced via a switching power supply. An electrode assembly is connected to the switching power supply and is submerged in the quantity of saltwater. The electrical current enters the user's bloodstream and circulates through the user's body, drawing toxins and other harmful substances as the electrical current exits the user's body.

### BRIEF DESCRIPTION OF THE DRAWINGS

FIG. 1 is a perspective view of the present invention.

FIG. 2 is a side view of the present invention.

FIG. 3 is a cross-sectional view of the present invention taken along line A-A from FIG. 2.

FIG. 4 is a detail view of the present invention taken from circle B of FIG. 3.

FIG. 5 is a top view of the present invention.

### DETAIL DESCRIPTIONS OF THE INVENTION

All illustrations of the drawings are for the purpose of describing selected versions of the present invention and are not intended to limit the scope of the present invention.

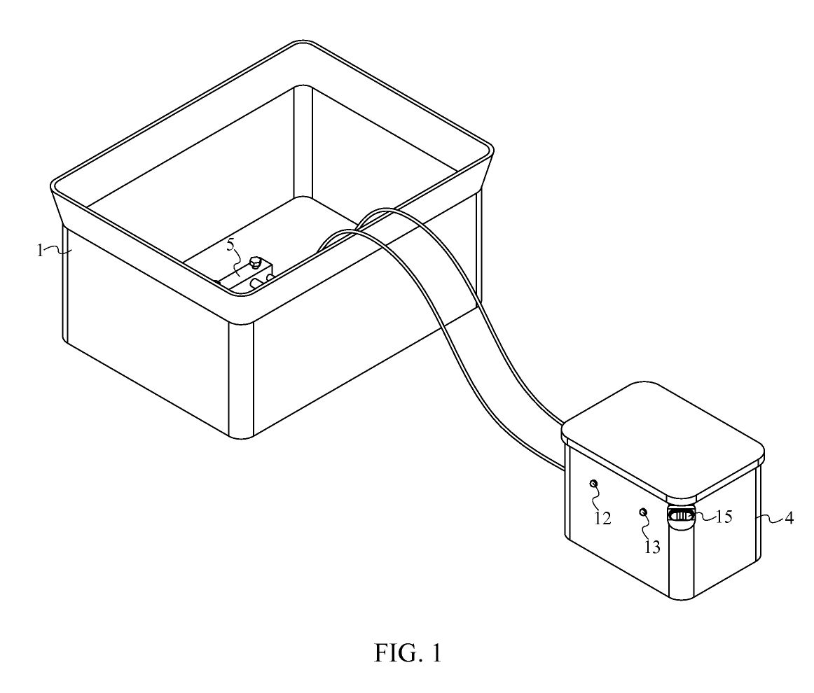

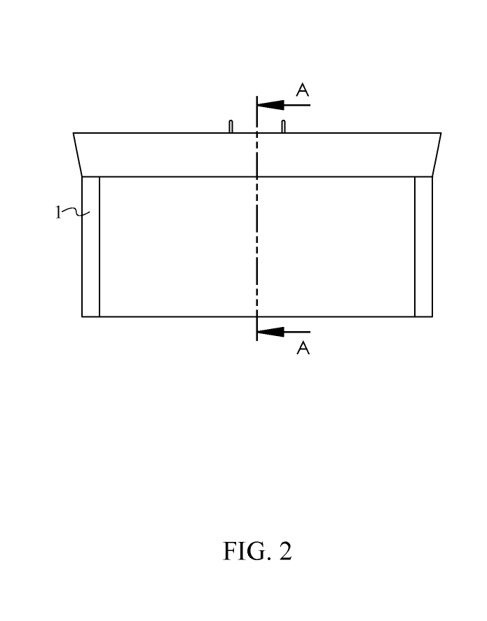

The present invention is an electron flow detoxification system that removes toxins and other harmful substances from the user's body utilizing electron flow through the user's body. The present invention is shown in FIGS. 1-4 and comprises a fluid reservoir 1, a switching power supply 2, a control unit 3, an enclosure 4, an electrode assembly 5, an electrical relay set 9, and a quantity of saltwater 10.

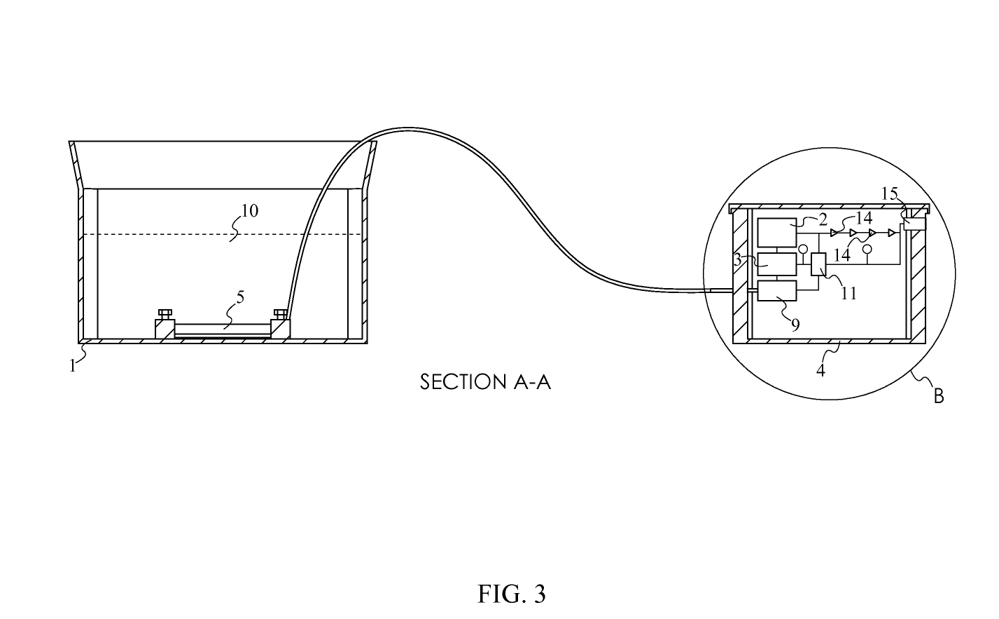

The fluid reservoir 1 is a container into which the user may place his or her hands or feet during use of the present invention. The fluid reservoir 1 is preferably able to hold a volume of approximately 18 qt. As shown in FIG. 3, the quantity of saltwater 10 is positioned within the fluid reservoir 1 and the user's hands or feet are submerged within the quantity of saltwater 10 during use of the present invention. The quantity of saltwater 10 contains approximately 2 oz of plain salt and approximately 1.5 gallons of water. The positively-charged sodium ions and the negatively-charged chlorine ions in the quantity of saltwater 10 are able to carry electricity through the quantity of saltwater 10.

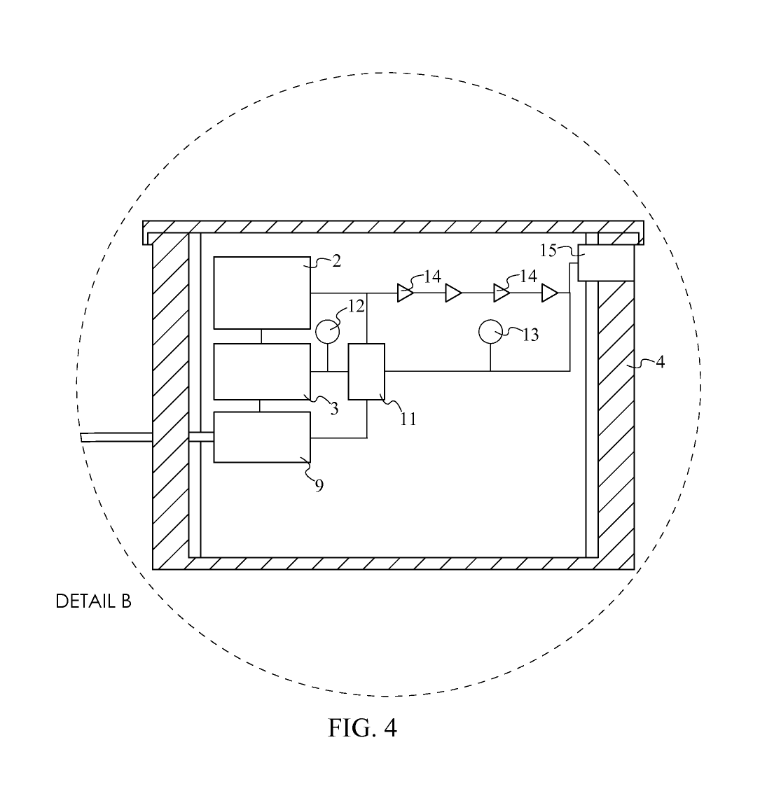

Again with reference to FIG. 3 and FIG. 4, the switching power supply 2 is connected to an energy source and provides electric energy to the present invention. The control unit 3 allows the user to perform various functions such as turning the switching power supply 2 on or off. As such, the control unit 3 is electrically connected to the switching power supply 2. The switching power supply 2 is preferably a 12 V power supply that is rated for at least 5 A. The electrical relay set 9 enables the user to reverse the electrical current flow direction through the users' body. The electrical current flow direction is reversed during use of the present invention in order to ensure that the user's pores do not become clogged. As such, the electrical current flow direction is reversed periodically while the present invention is in use. The enclosure 4 is a housing container for the electrical components of the present invention that are not intended to become wet. As such, the switching power supply 2, the control unit 3, and the electrical relay set 9 are positioned within the enclosure 4.

The electrode assembly 5 is a conductor through which electrical current is able to enter the nonmetallic medium provided by the quantity of saltwater 10. The electrode assembly 5 is submerged within the quantity of saltwater 10. This enables the electrical current to flow into the user's body when the switching power supply 2 is activated. The control unit 3 and the switching power supply 2 are electrically connected to the electrode assembly 5 through the electrical relay set 9. The electrical current provided by the switching power supply 2 is thus able to flow to the electrode assembly 5 before the electrical current enters the quantity of saltwater 10.

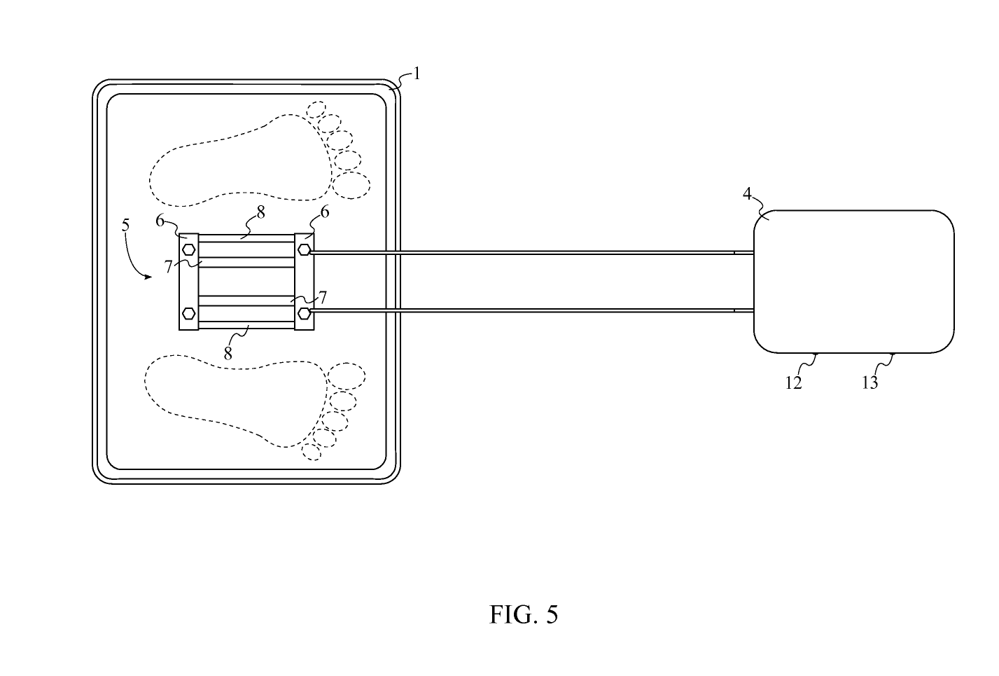

As shown in FIG. 5, the electrode assembly 5 comprises a pair of support members 6, a pair of electrodes 7, and a pair of protective guard members 8. The pair of support members 6 provide structural support to the electrode assembly 5 and are the components to which the pair of electrodes 7 and the pair of protective guard members 8 are mounted. The pair of electrodes 7 are the conductors through which the electrical current enters the quantity of saltwater 10. The pair of protective guard members 8 provide a physical obstruction to prevent the user's hands or feet from coming into contact with the pair of electrodes 7 while the present invention is in use. The pair of support members 6 are oriented parallel to each other. The pair of protective guard members 8 and the pair of electrodes 7 are connected in between the pair of support members 6. The pair of support members 6 are thus able to provide structural support to the pair of protective guard members 8 and the pair of electrodes 7. The pair of electrodes 7 are positioned in between the pair of protective guard members 8 to prevent the user's hands or feet from coming into contact with the pair of electrodes 7. The pair of protective guard members 8 are oriented parallel to the pair of electrodes 7 to physically prevent contact with the pair of electrodes 7 during use of the present invention. The pair of protective guard members 8 and the pair of electrodes 7 are oriented normal to the pair of support members 6, allowing the pair of support members 6 to provide structural support to both sides of the pair of protective guard members 8 and the pair of electrodes 7.

Again referring to FIG. 4, in the event that the electrical relay set 9 is not rated for use with the switching power supply 2, the present invention further comprises a voltage regulator 11. The voltage regulator 11 is able to maintain a constant voltage level. The fixed output voltage enables the use of the electrical relay set 9. For example, the voltage regulator 11 enables the use of an electrical relay set 9 that is rated up to 5 V with a 12 V switching power supply 2. The voltage regulator 11 is positioned within the enclosure 4 for protection during use of the present invention. The voltage regulator 11 is electrically connected to the switching power supply 2, the control unit 3, and the electrical relay set 9. This enables the voltage regulator 11 to output a constant voltage to the electrical relay set 9.

As shown in FIG. 1, FIG. 4, and FIG. 5, the present invention further comprises an electrode operational status notification light 12. The electrode operational status notification light 12 provides an indication to the user that electrical current is flowing through the electrode assembly 5. The electrode operational status notification light 12 is externally mounted to the enclosure 4 to allow the user to easily view the status of the electrode assembly 5. The electrode operational status notification light 12 is electrically connected to the electrode assembly 5. As such, when electrical current is flowing through the electrode assembly 5, the electrode operational status notification light 12 is turned on. Conversely, when there is no electrical current flowing through the electrode assembly 5, the electrode operational status notification light 12 is turned off.

In addition to the electrode operational status notification light 12, the present invention further comprises a system operational status notification light 13. The system operational status notification light 13 provides an indication to the user that the present invention is functional. The system operational status notification light 13 is externally mounted to the enclosure 4, allowing the user to easily see the system operational status notification light 13. The system operational status notification light 13 is electrically connected to the control unit 3 and is turned on when electrical power is available to the control unit 3. The system operational status notification light 13 is turned off when there is no electrical power available to the control unit 3.

The user is able to adjust the output voltage to the electrode assembly 5 if needed. This is particularly suitable if the user has sensitive hands or feet for which a particular voltage (e.g. 12 V) is not suitable. In this case, the present invention further comprises a plurality of diodes 14 and a voltage adjustment switch 15. The plurality of diodes 14 results in a voltage drop when the electrical current passes through the plurality of diodes 14. The voltage adjustment switch 15 allows the user to select the normal operating voltage or the lower voltage based on his or her needs during use of the present invention. The voltage adjustment switch 15 additionally allows the user to place the present invention into standby mode. The plurality of diodes 14 is electrically connected in series with each other in a cumulative voltage drop across the plurality of diodes 14. The number of diodes in the plurality of diodes 14 may vary according to the needs of the user. The voltage adjustment switch 15 is electrically connected to the plurality of diodes 14, the switching power supply 2, and the electrical relay set 9. As such, the voltage adjustment switch 15 may be utilized to adjust the output voltage before the electrical current passes through the electrical relay set 9 and reaches the electrode assembly 5.

In order to utilize the present invention, the user first submerges his or her hands or feet in the quantity of saltwater 10 and places them adjacent to the electrode assembly as shown in FIG. 5. The user's hands or feet do not come into contact with the pair of electrodes 7 during use of the present invention. The switching power supply 2 is turned on and an electrical current enters the quantity of saltwater 10. The electrical current enters the user's hands or feet and flows through the user's bloodstream. The electrical current then exits the user's body and draws out toxins and other harmful substances. The electrical relay set 9 may be utilized in order to reverse the electrical current flow direction and prevent the user's pores from clogging. The present invention is intended for use for a minimum of 45 minutes when detoxifying through the feet and a minimum of 25 minutes for detoxification through the hands after detoxifying through the feet. Alternatively, detoxification may be done solely through the hands for a minimum of 35 minutes.

Although the present invention has been explained in relation to its preferred embodiment, it is understood that many other possible modifications and variations can be made without departing from the spirit and scope of the present invention as hereinafter claimed.

What is claimed is:

1. An electron flow detoxification system comprises:
a fluid reservoir;
a switching power supply;
a control unit;
an enclosure;
an electrode assembly;
an electrical relay set;
a quantity of saltwater;
the quantity of saltwater being positioned within the fluid reservoir;
the electrode assembly being submerged within the quantity of saltwater;
the switching power supply, the control unit, and the electrical relay set being positioned within the enclosure;
the control unit being electrically connected to the switching power supply; and
the control unit and the switching power supply being electrically connected to the electrode assembly through the electrical relay set.

2. The electron flow detoxification system as claimed in claim 1 further comprises:
the electrode assembly comprises a pair of support members, a pair of electrodes, and a pair of protective guard members;
the pair of support members being oriented parallel to each other;
the pair of protective guard members and the pair of electrodes being connected in between the pair of support members;
the pair of electrodes being positioned in between the pair of protective guard members;
the pair of protective guard members being oriented parallel to the pair of electrodes; and
the pair of protective guard members and the pair of electrodes being oriented normal to the pair of support members.

3. The electron flow detoxification system as claimed in claim 1 further comprises:
a voltage regulator;
the voltage regulator being positioned within the enclosure; and
the voltage regulator being electrically connected to the switching power supply, the control unit, and the electrical relay set.

4. The electron flow detoxification system as claimed in claim 1 further comprises:
an electrode operational status notification light;
the electrode operational status notification light being externally mounted to the enclosure; and
the electrode operational status notification light being electrically connected to the electrode assembly.

5. The electron flow detoxification system as claimed in claim 1 further comprises:
a system operational status notification light;
the system operational status notification light being externally mounted to the enclosure; and
the system operational status notification light being electrically connected to the control unit.

6. The electron flow detoxification system as claimed in claim 1 further comprises:
a plurality of diodes;
a voltage adjustment switch;
the plurality of diodes being electrically connected in series with each other; and
the voltage adjustment switch being electrically connected to the plurality of diodes, the switching power supply, and the electrical relay set.

7. An electron flow detoxification system comprises:
a fluid reservoir;
a switching power supply;
a control unit;
an enclosure;
an electrode assembly;
an electrical relay set;
a quantity of saltwater;
a plurality of diodes;
a voltage adjustment switch;
the quantity of saltwater being positioned within the fluid reservoir;
the electrode assembly being submerged within the quantity of saltwater;
the switching power supply, the control unit, and the electrical relay set being positioned within the enclosure;
the control unit being electrically connected to the switching power supply;
the control unit and the switching power supply being electrically connected to the electrode assembly through the electrical relay set;
the plurality of diodes being electrically connected in series with each other; and
the voltage adjustment switch being electrically connected to the plurality of diodes, the switching power supply, and the electrical relay set.

8. The electron flow detoxification system as claimed in claim 7 further comprises:
the electrode assembly comprises a pair of support members, a pair of electrodes, and a pair of protective guard members;
the pair of support members being oriented parallel to each other;
the pair of protective guard members and the pair of electrodes being connected in between the pair of support members;
the pair of electrodes being positioned in between the pair of protective guard members;
the pair of protective guard members being oriented parallel to the pair of electrodes; and
the pair of protective guard members and the pair of electrodes being oriented normal to the pair of support members.

9. The electron flow detoxification system as claimed in claim 7 further comprises:
a voltage regulator;
the voltage regulator being positioned within the enclosure; and
the voltage regulator being electrically connected to the switching power supply, the control unit, and the electrical relay set.

10. The electron flow detoxification system as claimed in claim 7 further comprises:
an electrode operational status notification light;
the electrode operational status notification light being externally mounted to the enclosure; and
the electrode operational status notification light being electrically connected to the electrode assembly.

11. The electron flow detoxification system as claimed in claim 7 further comprises:
a system operational status notification light;
the system operational status notification light being externally mounted to the enclosure; and
the system operational status notification light being electrically connected to the control unit.

12. An electron flow detoxification system comprises:
a fluid reservoir;
a switching power supply;
a control unit;
an enclosure;
an electrode assembly;
an electrical relay set;
a quantity of saltwater;
a plurality of diodes;
a voltage adjustment switch;
a voltage regulator;
the quantity of saltwater being positioned within the fluid reservoir;
the electrode assembly being submerged within the quantity of saltwater;
the switching power supply, the control unit, and the electrical relay set being positioned within the enclosure;
the control unit being electrically connected to the switching power supply;
the control unit and the switching power supply being electrically connected to the electrode assembly through the electrical relay set;
the plurality of diodes being electrically connected in series with each other;
the voltage adjustment switch being electrically connected to the plurality of diodes, the switching power supply, and the electrical relay set;
the voltage regulator being positioned within the enclosure; and
the voltage regulator being electrically connected to the switching power supply, the control unit, and the electrical relay set.

13. The electron flow detoxification system as claimed in claim 12 further comprises:
the electrode assembly comprises a pair of support members, a pair of electrodes, and a pair of protective guard members;
the pair of support members being oriented parallel to each other;
the pair of protective guard members and the pair of electrodes being connected in between the pair of support members;
the pair of electrodes being positioned in between the pair of protective guard members;
the pair of protective guard members being oriented parallel to the pair of electrodes; and
the pair of protective guard members and the pair of electrodes being oriented normal to the pair of support members.

14. The electron flow detoxification system as claimed in claim 12 further comprises:
an electrode operational status notification light;
the electrode operational status notification light being externally mounted to the enclosure; and
the electrode operational status notification light being electrically connected to the electrode assembly.

15. The electron flow detoxification system as claimed in claim 12 further comprises:
a system operational status notification light;
the system operational status notification light being externally mounted to the enclosure; and
the system operational status notification light being electrically connected to the control unit.
